(19) United States
(12) Patent Application Publication
(10) Pub. No.: US 2026/0142018 A1
(43) Pub. Date: May 21, 2026

(54) SYSTEM AND METHOD FOR IMPROVING BRAIN HEALTH IN A VIRTUAL REALITY ENVIRONMENT

(71) Applicant: ARC VRX LLC, Frisco, TX (US)

(72) Inventor: Shawnee Jean Harkins, Frisco, TX (US)

(21) Appl. No.: 18/951,076

(22) Filed: Nov. 18, 2024

(51) Int. Cl. G16H 20/70

(52) U.S. Cl. CPC G16H 20/70

(57) ABSTRACT

The disclosed principles provide a system and method for improving brain health in a virtual reality environment. The system includes a virtual reality appliance and an artificial intelligence enabled program for providing and customizing the virtual reality environment with a treatment plan. The artificial intelligence enabled program includes an initial treatment phase wherein a user's baseline metrics are measured. Using these metrics, a treatment plan is created during a treatment plan development phase. The treatment plan is implemented during a treatment phase through a series of repeated mindfulness, cognitive, and physical exercises presented in a virtual reality environment. Data corresponding to the user's inputs during treatment is collected and stored. An artificial intelligence program analyzes user input data and modifies the treatment plan accordingly to optimize treatment. Treatment and evaluation and modification are alternately repeated according to the personalized, artificial intelligence-modified treatment plan to strengthen neural pathways.

### TECHNICAL FIELD

This disclosure relates generally to the field of neurorehabilitation, and more particularly to a novel system and method for improving brain health in a virtual reality environment.

### BACKGROUND

Traumatic brain injury, stroke, and dementia are neurological conditions associated with damaged brain tissue. FIG. 1 is a schematic side cross-sectional view of a human head with damaged brain tissue. The brain 102 is surrounded by protective meninges 104 filled with cerebrospinal fluid, and the skull 106. While operating by different mechanisms, traumatic brain injury, stroke, and dementia each cause areas of damaged brain tissue 108. Damaged brain tissue 108 disrupts normal brain function by impairing the transmission of electrical signals between neurons, the functional cells of the brain. This can lead to a wide range of symptoms, including cognitive deficits, motor impairments, and emotional instability.

Traumatic brain injury (TBI) results when external physical forces impact the head with sufficient intensity to cause damage to the brain. About 2.8 million individuals sustain a TBI each year in the United States, and global rates of TBI have increased over the last three decades. FIG. 2 illustrates the number of emergency department visits, hospitalizations, and deaths due to TBI each year in the United States. TBI can impair cognition, behavior, emotion, and motor function, and is associated with long-term symptoms including impaired memory, attention, learning, and coordination, headache, and mood disorders. An estimated 5 million individuals are living with TBI-related disability in the United States. TBI is also associated with reduced lifespan, and there has been no notable change in long-term survival over the past three decades.

Stroke occurs when there is a prolonged disruption of blood flow to the brain, leading to cell death and neurological impairment. Approximately 795,000 people in the United States experience a stroke each year, and the incidence rate has increased over the last decade. FIG. 3 illustrates the prevalence of stroke in the United States each year, as well as the projected prevalence by the end of the decade. Stroke can result in significant impairments in speech, mobility, and cognitive function, leading to long-term disabilities in over two-thirds of stroke survivors. Additionally, stroke is associated with a loss of 5.5-7.5 years of life expectancy.

Dementia refers to a group of cognitive disorders that are primarily characterized by memory loss. Dementia is associated with various neurophysiological changes such as the accumulation of amyloid plaques, reduced tissue volume, inflammation, and changes in blood flow. In the United States, about 6.9 million people aged 65 and older are living with dementia. This number is projected to rise significantly as the population ages. FIG. 4 illustrates the projected prevalence of dementia in people aged 65 and older in the United States. Dementia is associated with cognitive, behavioral, and emotional impairment, and long-term symptoms including memory loss, difficulty reasoning, impaired communication, disorientation, and mood and behavior changes. Compared to the general population, people diagnosed with dementia have a reduced life expectancy of 4-10 years.

Neurorehabilitation is often utilized in treatment of each of these conditions. Traditional neurorehabilitation involves several distinct therapies, including physical therapy (e.g., balance, mobility, and strength training), cognitive therapy (e.g., memory, attention, and problem-solving training), psychological therapy, occupational therapy, and speech and language therapy. While these therapies are beneficial, conventional neurorehabilitation has several shortfalls including limited personalization of treatment, difficulty in maintaining patient engagement, reliance on in-person sessions that may not be accessible or convenient, and a lack of real-time feedback or adaptive therapies.

Virtual reality is an immersive technology that simulates realistic environments through computer-generated imagery, audio, and physical stimuli, allowing users to interact with and explore virtual spaces using specialized equipment. Virtual reality has found a wide range of applications, from immersive gaming to medical and military training. In the field of healthcare, virtual reality provides a unique platform for patients to engage in therapeutic scenarios in a controlled and safe environment.

Considering the increasing prevalence of debilitating neurological conditions associated with damaged brain tissue, the shortfalls of conventional neurorehabilitation, and the therapeutic functionalities of virtual reality, what is needed in the art is a system and method to improve brain health in the virtual reality environment.

### SUMMARY

Novel aspects of the present disclosure are directed to a system for improving brain health in a virtual reality environment. In one embodiment, the system includes a virtual reality appliance and an artificial intelligence enabled program for providing and customizing the virtual reality environment with a treatment plan. The artificial intelligence enabled program includes an initial testing phase, a treatment development phase, a treatment phase, and an evaluation and modification phase. During the initial testing phase, the user's baseline metrics are measured using a virtual reality appliance. The user's inputs are then used to develop a treatment plan. The treatment plan is implemented during the treatment phase through a series of exercises presented in a virtual reality environment in accordance with The Harkins Method, Neural Network of Change. The artificial intelligence program analyzes data collected during the treatment phase and modifies the treatment plan accordingly to prescribe optimized physical and cognitive exercises in a virtual reality environment. Treatment is then repeated according to the personalized, artificial intelligence-modified treatment plan to form and strengthen new neural pathways.

Novel aspects of the present disclosure are also directed to a method for improving brain health in the virtual reality environment. The method includes initial testing, wherein the user's baseline metrics are measured using a virtual reality appliance. An initial treatment plan is developed by comparing the user's metrics to the average. During treatment, the treatment plan is implemented through a series of exercises presented in a virtual reality environment in accordance with The Harkins Method, Neural Network of Change. Treatment includes a neurochemical balance phase, wherein excitatory and inhibitory neurotransmitter levels are reset to normal levels. Treatment also includes an oxygenation reset phase, wherein circulation and oxygenation to the brain and muscles is increased. Treatment also includes an activity phase, wherein the user is engaged in a series of repeated virtual reality experiences. Data corresponding to the user's inputs is collected and stored. An artificial intelligence program analyzes data collected during the treatment phase and modifies the treatment plan accordingly to optimize treatment. Treatment is then repeated according to the personalized, artificial intelligence-modified treatment plan to form and strengthen new neural pathways.

Additional embodiments and advantages and variation thereof are also encompassed within the scope of the disclosed principles, and some such exemplary embodiments are discussed in further detail herein.

### BRIEF DESCRIPTION OF THE DRAWINGS

The novel features believed characteristic of the disclosure are set forth in the appended claims. The disclosure itself, however, as well as a preferred mode of use, further objectives, and advantages thereof, will be best understood by reference to the following detailed description of illustrative embodiments when read in conjunction with the accompanying drawings, in which:

FIG. 1 illustrates a patient with damaged brain tissue;

FIG. 2 is graph depicting the number of emergency department visits, hospitalizations, and deaths due to traumatic brain injury each year in the United States;

FIG. 3 is graph depicting the prevalence of stroke in the United States each year;

FIG. 4 is graph depicting the projected prevalence of dementia among adults aged 65 and older in the United States;

FIG. 5 illustrates a user wearing an exemplary virtual reality appliance with a visualization screen and two input controllers in accordance with the present disclosure;

FIG. 6 illustrates an exemplary scene depicted in the user's visual display in accordance with the present disclosure;

FIG. 7 is a flowchart of a process for improving brain health in a virtual reality environment in accordance with the present disclosure;

FIG. 8 is a block diagram of the initial testing phase of a process for improving brain health in a virtual reality environment in accordance with the present disclosure;

FIG. 8A illustrates an exemplary scene depicted in a user's visual display for testing the user's physical capabilities in accordance with the present disclosure;

FIG. 8B illustrates another exemplary scene depicted in a user's visual display for testing the user's physical capabilities in accordance with the present disclosure;

FIG. 8C illustrates an exemplary scene depicted in a user's visual display for testing the user's cognitive capabilities in accordance with the present disclosure;

FIG. 8D illustrates another exemplary scene depicted in a user's visual display for testing the user's cognitive capabilities in accordance with the present disclosure;

FIG. 8E illustrates another exemplary scene depicted in a user's visual display for measuring the user's mental state, emotional condition, and pain level;

FIG. 9 is a block diagram of the treatment plan development process in accordance with the present disclosure;

FIG. 10 is a block diagram illustrating the treatment step of a process for improving brain health in a virtual reality environment in accordance with the present disclosure;

FIG. 10A illustrates an exemplary scene depicted in the user's visual display for the neurochemical balance phase of treatment in accordance with the present disclosure;

FIG. 10B illustrates an exemplary scene depicted in the user's visual display for the oxygenation reset phase of treatment in accordance with the present disclosure;

FIG. 10C illustrates an exemplary scene depicted in the user's visual display for the activity phase of treatment in accordance with the present disclosure;

FIG. 10D illustrates an exemplary scene depicted in the user's visual display for the activity phase of treatment in accordance with the present disclosure;

FIG. 10E illustrates an exemplary scene depicted in the user's visual display for an initial and concluding well-being assessment in accordance with the present disclosure;

FIG. 11 is a diagram illustrating the key components of the treatment step of a process for improving brain health in a virtual reality environment in accordance with the present disclosure;

FIG. 12 illustrates a block diagram of the evaluation and modification process in accordance with the present disclosure;

FIG. 13 illustrates a block diagram of the training management system and portal in accordance with the present disclose.

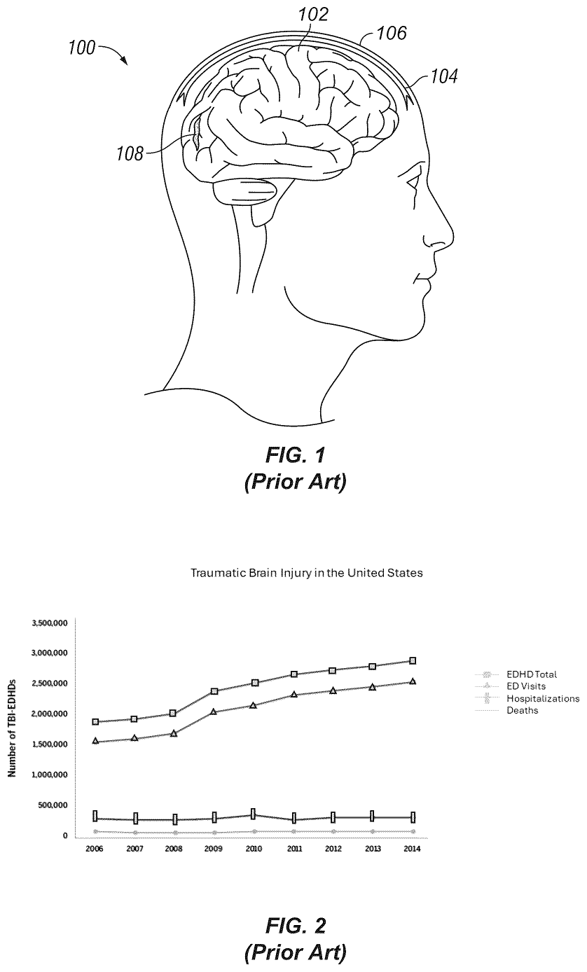

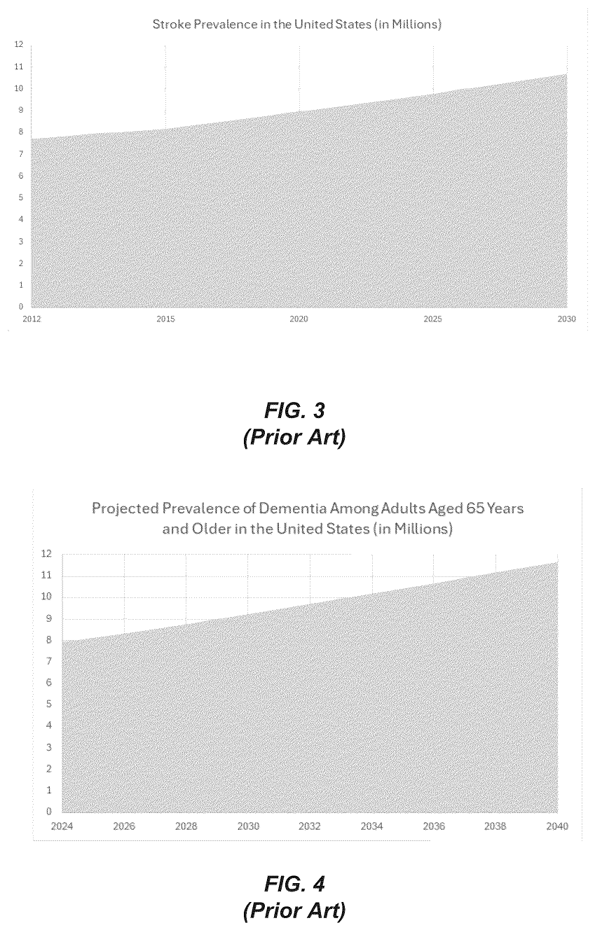

### DETAILED DESCRIPTION

For the purpose of promoting an understanding of the principles in the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein, are contemplated as would normally occur to one skilled in the art to which the invention relates. Although multiple embodiments are shown and discussed in detail, it will be apparent to those skilled in the relevant art that some features that are not relevant to the present invention may not be shown for the sake of clarity.

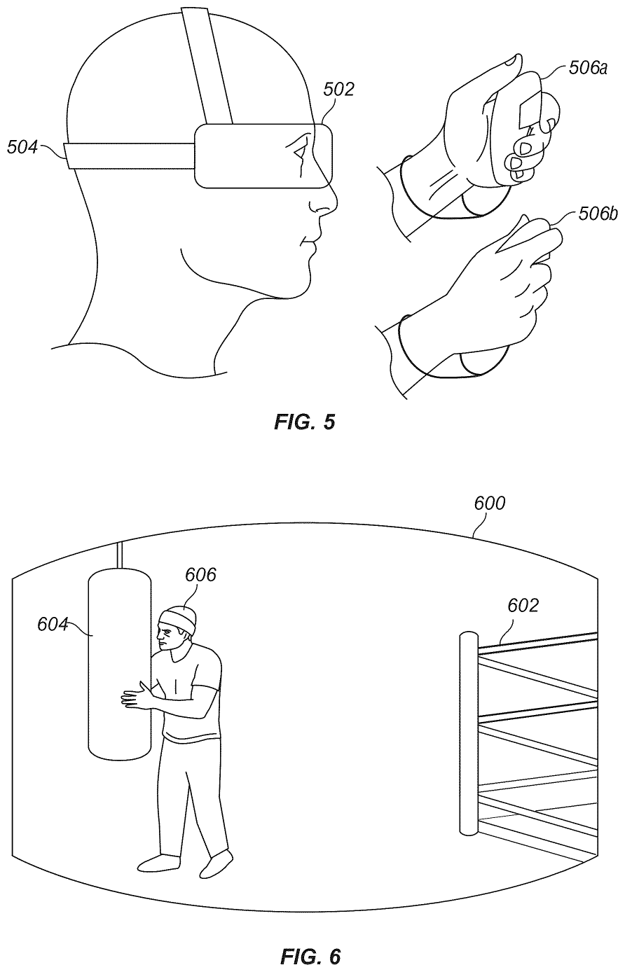

FIG. 5 illustrates a user with an exemplary virtual reality appliance with a visualization screen and two input controllers. In this non-limiting embodiment, the visualization screen 502 is a wearable headset that covers the user's eyes for visual immersion. The visualization screen 502 can display a two-dimensional or three-dimensional representation of the virtual environment. The visualization screen 502 may include straps 504 to comfortably secure the visualization screen 502 to the user's head. The headset may also include motion sensors to detect and track movement of the user's eyes and head. The headset may also include speakers to facilitate the delivery of auditory stimuli. In the non-limiting embodiment illustrated in FIG. 5, the input controllers 506a and 506b are handheld devices that allow the user to interact with the virtual reality environment. The input controllers 506a, 506b may include sensors to detect movement. Movement data is collected and translated into actions within the virtual reality environment. Input controllers 506a, 506b may also include buttons to facilitate user interaction with the virtual environment. The input controllers 506a, 506b may also include vibration motors and actuators for the delivery of physical stimuli such as haptic feedback. Many other embodiments of visualization screens 502 and input controllers 506a, 506b that can achieve the same utility are within the scope of the claims.

FIG. 6 illustrates an exemplary scene depicted in the user's visual display for testing the user's physical and cognitive capabilities. In this non-limiting exemplary scene 600, the user's display depicts a boxing ring 602 with a hanging punching bag 604 and a trainer avatar 606 in a gym. The trainer avatar 606 may be generated from volumetric capture data of the developer, Shawnee Harkins. Alternatively, the trainer avatar 606 may be a computer-generated avatar. The trainer avatar 606 may introduce and explain the Harkins Method, Neural Network of Change described in FIG. 12. The trainer avatar 606 may prompt the user to interact with the virtual reality environment. The user may interact with the scene by moving the input controllers 506a, 506b described in FIG. 5. While exemplary scenes that may be depicted in the user's visual display are described in detail below, it should be recognized that the system is not limited to any particular virtual environment.

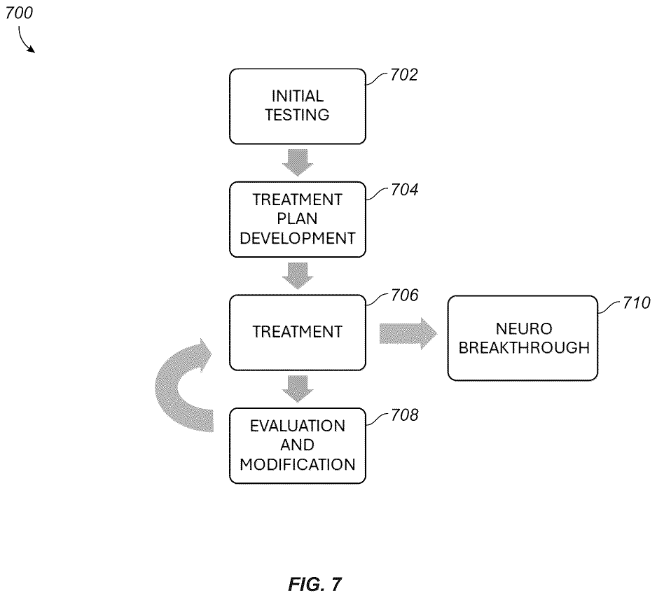

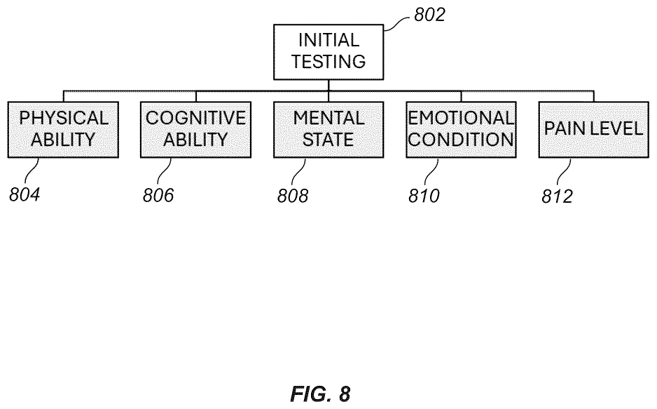

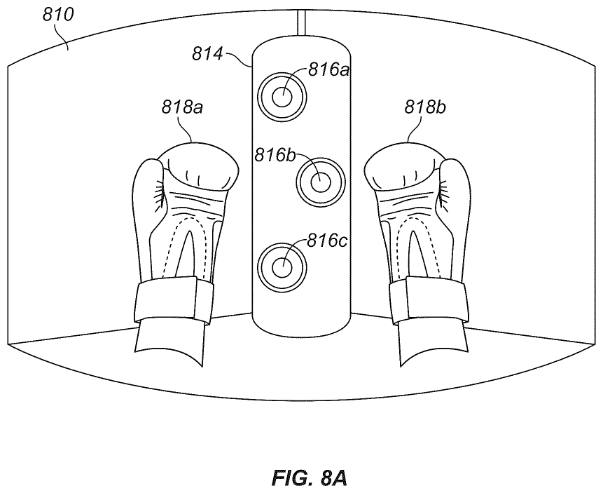

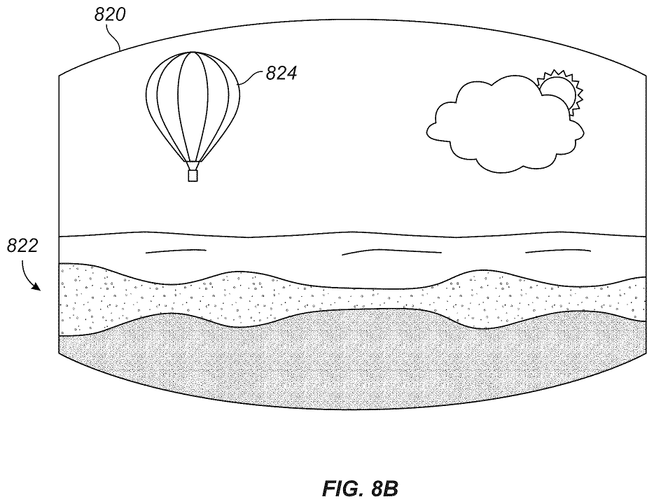

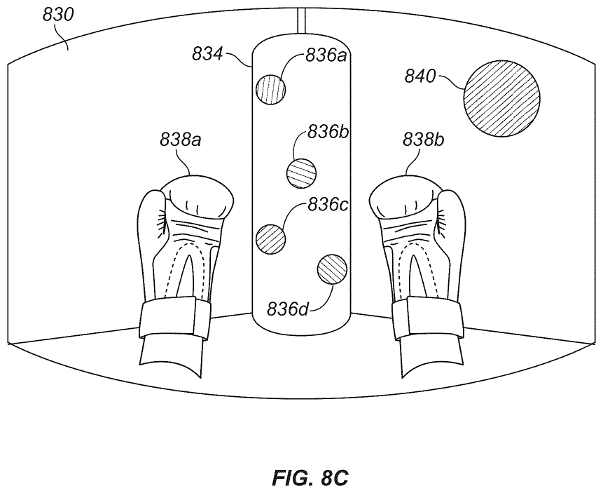

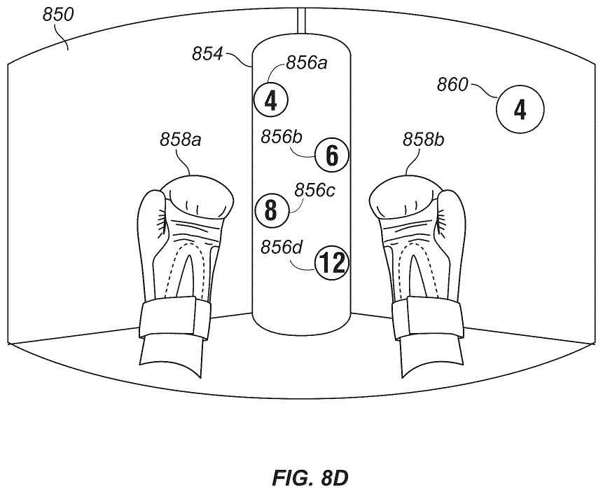

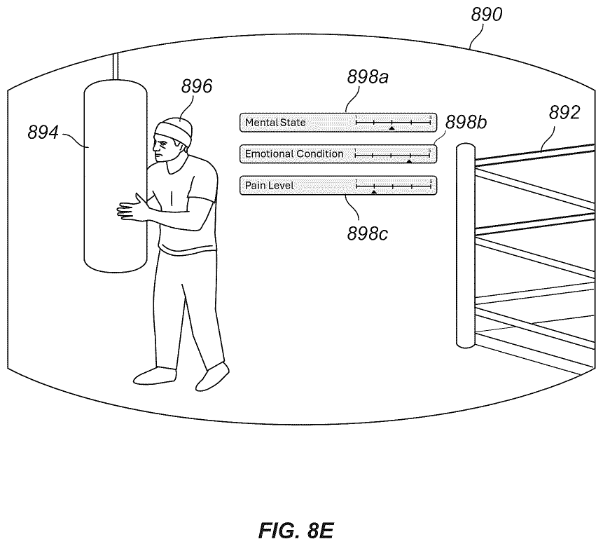

FIG. 7 is a flowchart of a process for improving brain health in a virtual reality environment. The steps of flowchart 700 can be implemented using a virtual reality appliance, such as the virtual reality appliance illustrated in FIG. 5. Flowchart 700 begins with initial testing 702, wherein the user's baseline metrics are measured using a virtual reality appliance. During initial testing 702, the user may be presented with several exercises within the virtual reality environment. That is, a series of scenes can be depicted on the visualization screen 502 and the user may be prompted to interact with these scenes using the input controllers 506a, 506b illustrated in FIG. 5. Data generated from the user's input using the input controllers 506a, 506b may be collected and stored. The components of initial testing 702 are discussed in more detail in FIG. 8.

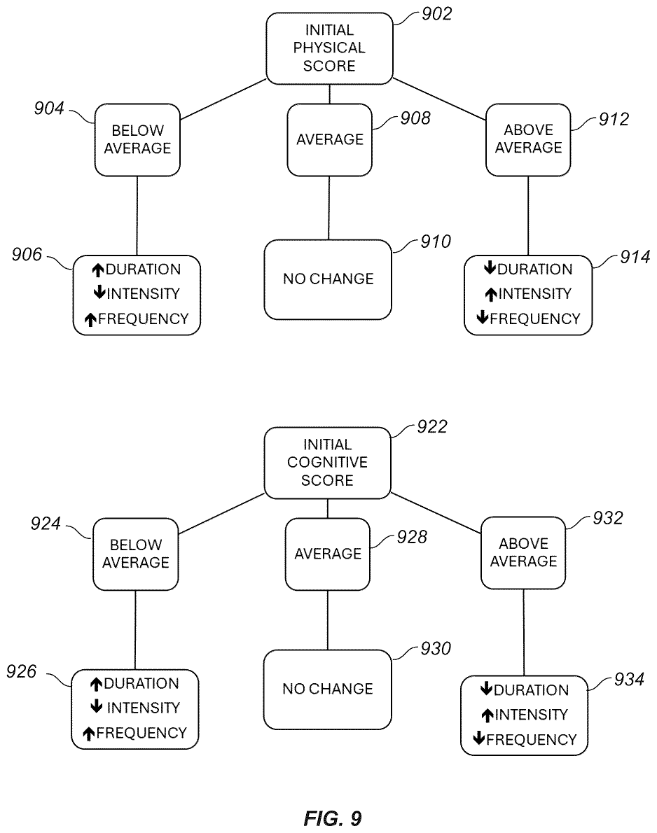

The next step is treatment plan development 704. The treatment plan is initially developed using the data collected from initial testing 702. The treatment plan prescribes the optimal intensity, duration, and frequency of physical and cognitive exercises to improve brain health in a virtual reality environment in accordance with The Harkins Method, Neural Network of Change. The components of treatment plan development 704 are discussed in more detail in FIG. 9.

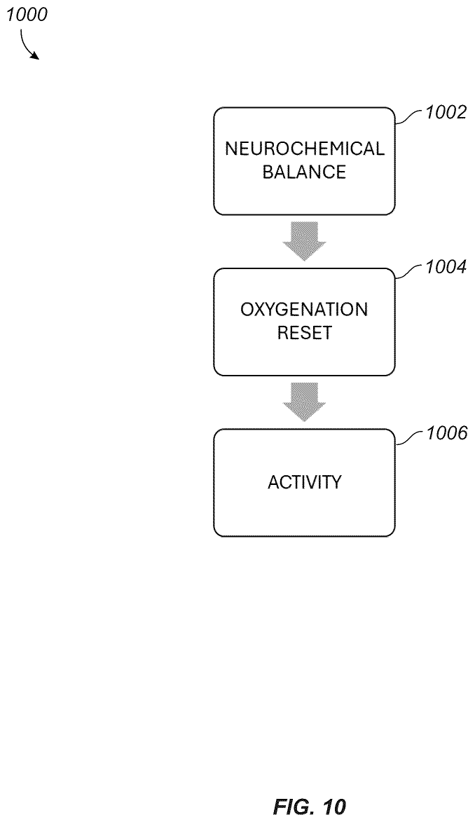

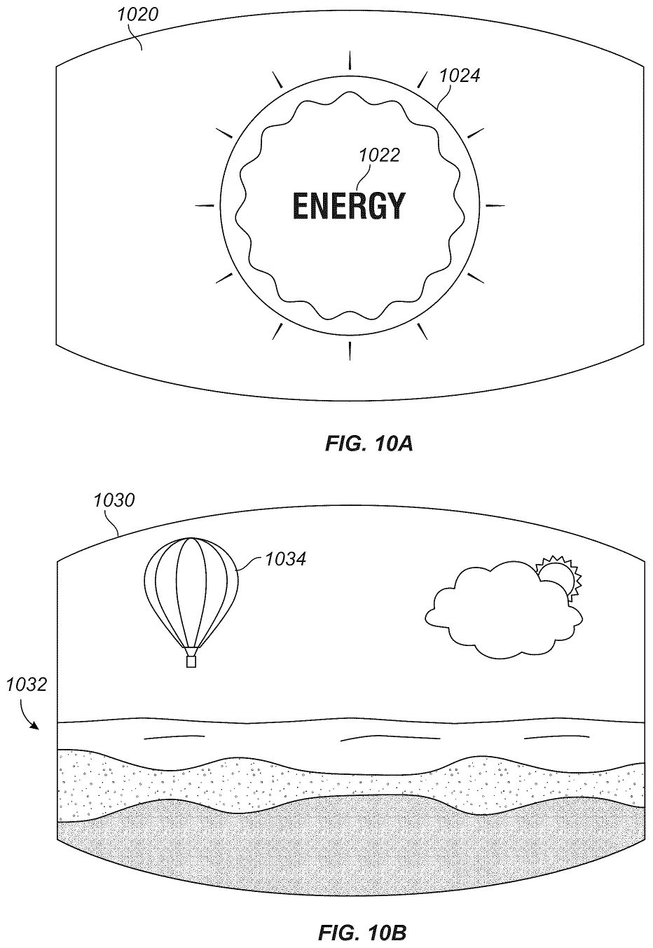

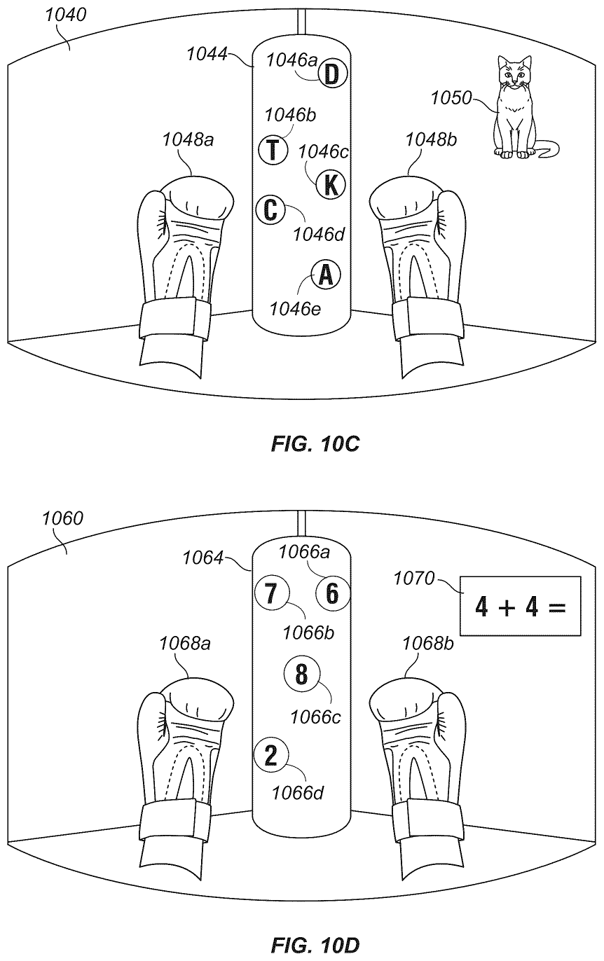

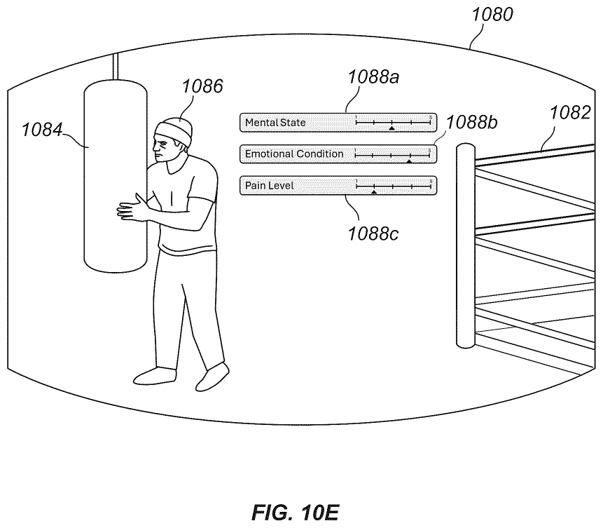

The third step in flowchart 700 is treatment 706. During initial treatment 706, the treatment plan created during treatment plan development 704 may implemented. During subsequent treatments 706, a modified treatment plan created during evaluation and modification 708 (discussed below) may be implemented. A user may be presented with various visual, auditory, and physical stimuli within the virtual reality environment and may be prompted to interact with virtual reality scenes using the input controllers 506a, 506b depicted in FIG. 5. Data generated from the user's inputs using the input controllers 506a, 506b is collected and stored. The components of treatment 706 are discussed in more detail in FIG. 10.

The next step is evaluation and modification 708. During this step, artificial intelligence is used to evaluate the data generated by the user's input during treatment 706 and modify the treatment plan to optimize the improvement of brain health in a virtual reality environment. This modified treatment plan is then implemented in the subsequent treatment 706 session. Repetition of treatment 706 and evaluation and modification 708 according to a personalized, artificial intelligence-generated treatment plan leads to a neuro-breakthrough 710, wherein new neural pathways are formed and strengthened. The components of evaluation and modification 708 are discussed in more detail in FIG. 12.

FIG. 8 is a block diagram depicting the metrics gathered during initial testing. In the non-limiting example, initial testing 802 includes collecting data regarding the user's physical ability 804 cognitive ability 806. While physical ability 804 corresponds to the user's ability to move within the virtual reality environment, cognitive ability 806 is associated with the user's ability to solve problems presented in the virtual reality environment. The user's physical ability 804 and cognitive ability 806 are determined from the user's input data collected during a series of exercises within the virtual reality environment. Exemplary exercises are described in detail in FIGS. 8A-8D. Initial testing 802 also includes collecting data regarding the user's mental state 808, emotional condition 810, and pain level 812. The user's mental state 808, emotional condition 810, and pain level 812 are determined from the user's input data collected in response to a series of questions presented within the virtual reality environment. An exemplary scene depicted in the user's display for collecting data regarding the user's mental state 808, emotional condition 810, and pain level 812 is described in FIG. 8F. While measurement of exemplary baseline metrics has been described, it should be recognized that measurement of other baseline metrics is within the scope of the claims.

FIG. 8A illustrates an exemplary scene depicted in the user's visual display for testing the user's physical capabilities. In this non-limiting exemplary scene 810, the user's display depicts a hanging punching bag 814 featuring various targets 816a, 816b, 816c. From the first-person perspective, the scene depicts the user inside the boxing ring wearing boxing gloves 818a and 818b. The user is instructed to punch each target 816a, 816b, 816c on the punching bag 814 using the input controllers 506a, 506b illustrated in FIG. 5. Data corresponding to the user's inputs, such as the strength, speed, and accuracy of each action, is collected and stored. This data can be evaluated to measure the user's physical abilities.

FIG. 8B illustrates another exemplary scene depicted in the user's visual display for testing the user's physical capabilities. In this non-limiting exemplary scene 820, the user's display depicts a beach 822 with a balloon 824 floating in the distance. From the first-person perspective, the scene depicts the user on the beach 822. The user is instructed to engage in forceful expiration to inflate the balloon 824. Data including lung volume and flow rates can be collected and stored. This data can be evaluated to measure the user's physical abilities.

FIG. 8C illustrates an exemplary scene depicted in the user's visual display for testing the user's cognitive capabilities. In this non-limiting exemplary scene 830, the user's display depicts a hanging punching bag 834. The scene 830 also depicts a first-person perspective of the user inside the boxing ring 832 wearing boxing gloves 838a, 838b. In this non-limiting embodiment, the punching bag 834 includes targets 836a, 836b, 836c, 836d of various colors. A test color 840 is displayed to the user. The user is instructed to punch the target 836c that matches the displayed test color 840 using the input controllers 506a, 506b illustrated in FIG. 5. Data corresponding to the accuracy of the user's inputs is collected and stored. This data can be evaluated to measure the user's cognitive abilities.

FIG. 8D illustrates another exemplary scene depicted in the user's visual display for testing the user's cognitive capabilities. In this non-limiting exemplary scene 850, the user's display depicts a hanging punching bag 854 featuring targets 856a, 856b, 856c, 856d depicting various numbers. From the first-person perspective, the scene depicts the user inside the boxing ring 852 wearing boxing gloves 858a, 858b. A test number 860 is displayed to the user. The user is instructed to punch the target 856a that matches the displayed test number 860 using the input controllers 506a, 506b illustrated in FIG. 5. Data corresponding to the accuracy of the user's inputs is collected and stored. This data can be evaluated to measure the user's cognitive abilities.

Additional scenes may be depicted in the user's visual display for testing the user's physical capabilities. For example, the user's display may depict the trainer avatar in a boxing ring wearing punching mitts. From the first-person perspective, the scene can depict the user inside the boxing ring wearing boxing gloves. A target hitting pattern may be displayed to the user. The user may be instructed to punch the trainer avatar's punching mitts according to the target hitting pattern using the input controllers 506a, 506b illustrated in FIG. 5. Data corresponding to the accuracy of the user's inputs is collected and stored. This data can be evaluated to measure the user's physical abilities.

FIG. 8E illustrates an exemplary scene depicted in the user's visual display for collecting data regarding the user's mental state, emotional condition, and pain level. In this non-limiting exemplary scene 890, the user's display depicts a boxing ring 892 with a hanging punching bag 894 and a trainer avatar 896 in a gym. The user's display may also present a series of interactive prompts 898a, 898b, 898c for measuring the user's mental, emotional, and pain status. For example, the user's display may depict a mental state prompt 898a, an emotional condition prompt 898b, and a pain level prompt 898c. In the non-limiting example illustrated in FIG. 8F, the prompts 898 a, 898 b, 898 c include a movable scale ranging from 1-5. The user can be instructed to respond to the prompts 898a, 898b, 898c using input controllers 506a, 506b illustrated in FIG. 5. For example, the user can be instructed to indicate their mental state by using the input controllers 506a, 506b to select a number along the continuum presented in the mental state prompt 898a. User input data can be collected and stored for evaluation during treatment plan development.

FIG. 9 is a block diagram of the treatment plan development process. The treatment plan is developed by comparing the user's initial physical and cognitive scores to average physical and cognitive scores. The average physical and cognitive scores can be adjusted for factors that impact brain health improvement following damage to brain tissue. Example factors include but are not limited to age, injury-severity, and time to treatment. Other baseline metrics including but not limited to mental state, emotional condition, and pain level can also be considered during treatment plan development. Treatment plan development can be completed by a qualified professional or, alternatively, by an artificial intelligence enabled program.

Beginning with the initial physical score 902, the user's initial physical score 902 is calculated based on a user's performance on physical exercises (e.g., FIGS. 8A and 8B) during initial testing. The user's initial physical score 902 is compared to an average physical score. If the initial physical score 902 is below average 904, the physical component 906 of the treatment plan will feature physical exercises with a lower intensity, longer duration, and increased frequency compared to initial testing. If the initial physical score is average 908, the physical component 910 of the treatment plan will feature physical exercises with the same intensity, duration, and frequency as the initial testing phase. If the initial physical score is above average 912, the physical component 914 of the treatment plan will feature physical exercises with a higher intensity, shorter duration, and lower frequency compared to initial testing.

The user's initial cognitive score 922 is also compared to the average cognitive score. The user's initial cognitive score 922 is calculated based on a user's performance on cognitive exercises (e.g., FIGS. 8C and 8D) during initial testing. If the initial cognitive score 922 is below average 924, the cognitive component 926 of the treatment plan will feature cognitive exercises with a lower intensity, longer duration, and higher frequency compared to initial testing. If the initial cognitive score is average 928, the cognitive component 930 of the treatment plan will feature cognitive exercises with the same intensity, duration, and frequency as the initial testing phase. If the initial cognitive score is above average 932, the cognitive component 934 of the treatment plan will feature cognitive exercises with a higher intensity, shorter duration, and lower frequency compared to initial testing.

Mental, emotional, and pain level initial scores (not shown) are also considered in treatment plan development. For example, if the initial mental or emotional score is low or the initial pain score is high, the treatment plan may feature exercises of lower intensity and shorter duration. The initial mental, emotional, and pain level scores can also inform the proportional duration of each phase of treatment (discussed below). For example, if the initial mental or emotional score is low or the initial pain score is high, the treatment plan may feature more neurochemical balance and oxygenation reset phase activities, and fewer activity phase exercises.

All components measured during initial testing are combined to develop a comprehensive treatment plan, wherein physical and cognitive therapies are integrated to optimize improvement of brain health in a virtual reality environment. The treatment plan is administered during the treatment phase of the process for improving brain health in a virtual reality environment.

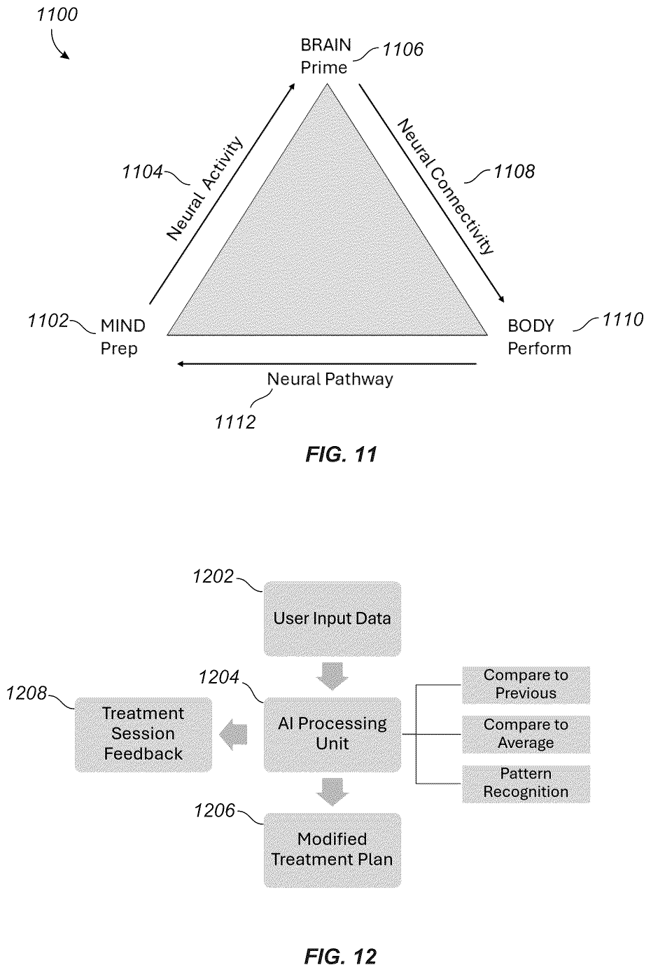

FIG. 10 is a flowchart of the treatment step of a process for improving brain health in a virtual reality environment. The treatment step can be implemented in accordance with The Harkins Method, Neural Network of Change discussed in FIG. 11. During each phase of treatment, the user may be presented with various scenes within the virtual reality environment and may be prompted to interact with the scenes using the input controllers 506a, 506b depicted in FIG. 5. The user may also be presented with various auditory and physical stimuli within the virtual reality environment during treatment.

Flowchart 1000 begins with a neurochemical balancing phase 1002, wherein excitatory and inhibitory neurotransmitter levels are reset to normal levels. The ability of neurons to respond to inputs is finely controlled through the balance of excitatory and inhibitory neurotransmitters. The excitatory-inhibitory balance is central to maintaining neural firing and inducing neuroplasticity. Patients with traumatic brain injury, stroke, and dementia often experience excitatory-inhibitory imbalance. As such, restoration of the excitatory-inhibitory balance is key to improving brain health in patients suffering from these neurological impairments. During the neurochemical balance phase 1002, visual, auditory, and physical stimuli may be presented to the user to encourage restoration of excitatory-inhibitory balance. Beginning with visual stimuli, the user may be guided through a virtual reality environment designed to enable visualization of neural firing and connectivity, and neurotransmitter balance. An exemplary scene that can be depicted on a user's visual display during the neurochemical balance phase 1002 is illustrated in FIG. 10A. The neurochemical balance phase 1002 may also include audio therapy, wherein auditory stimuli corresponding to the visual stimuli are presented to the user. For example, the user may be presented with simultaneous auditory stimuli of slightly different frequencies to create binaural beats and stimulate brain activity. Other examples of auditory stimuli that can be presented to the user during the neurochemical balance phase 502 include but are not limited to nature sounds, light classical music, and sounds or music embedded with delta or theta waves to support neuroplasticity. Other examples of auditory stimuli include but are not limited to binaural beats, nature sounds, and light classical music. The neurochemical balance phase 1002 may also include physical stimuli administered through the input controllers 506a, 506b illustrated in FIG. 5. For example, haptic vibration corresponding to the visual and auditory stimuli may be sent to the input controllers 506a, 506b. The neurochemical balance phase 1002 may also include guided meditation, mindfulness exercises, breathwork, and positive affirmations.

The next step is an oxygenation reset phase 1004, wherein circulation and oxygenation to the brain and muscles is increased. The major pathogenic mechanisms of TBI, stroke, and dementia each include low blood flow and poor oxygenation in brain tissue. Improved circulation and oxygenation have been shown to aid in reducing oxidative stress, promoting cellular repair, and enhancing neuroplasticity. During oxygenation reset 1004, the user is presented with a virtual reality exercise designed to encourage deep, controlled breathing. This type of breathing exercise serves to regulate blood pressure, heart rate, and circulation and oxygenation to the brain and muscles. An exemplary scene that can be depicted on a user's visual display during the oxygenation reset phase 1004 is illustrated in FIG. 10B.

The third step is an activity phase 1006. During the activity phase 1006, the user is engaged in a series of repeated experiences according to a personalized, artificial intelligence-generated formula to facilitate continual neural activity. This repeated neural activity initiates neural connectivity, resulting in the development of new neuropathways through repetitive hardwiring of these experiences. The experiences may incorporate a wide variety of topics, including but not limited to fitness, nutrition, cognition, emotional regulation, spiritual wellness, communication, and education. Experiences include but are not limited to math, spelling, vocabulary, word recall, sentence structure, reading and writing, strength, flexibility, mobility, executive functioning, and speech. Exemplary scenes that can be depicted on the user's visual display during the activity phase 1006 are illustrated in FIG. 10C and FIG. 10D.

The treatment step of a process for improving brain health in a virtual reality environment may also include initial and concluding well-being assessments (not shown) wherein the user's mental state, emotional condition, and pain level are examined. Well-being assessments improve user mindfulness and motivation and provide additional user input data for evaluation and modification of the treatment plan. An exemplary scene depicted in the user's display during initial and concluding well-being assessments is illustrated in FIG. 10E.

FIG. 10A illustrates an exemplary scene depicted in the user's visual display for the neurochemical balance phase of treatment. In this non-limiting exemplary scene 1020, the user's display depicts an empowering word 1022 inside a glowing, vibrating circle 1024. Using the input controllers 506a, 506b illustrated in FIG. 5, the user can select their preferred empowering word 1022. In the non-limiting exemplary scene depicted in FIG. 10A, the user's display depicts the word “energy.” The user may be engaged in guided meditation, mindfulness exercises, and positive affirmations centered on the empowering word 1022. For example, the user may be instructed to mentally or verbally repeat the empowering word 1022. In another example, the user may be instructed to personalize the empowering word 1022. The color of the circle 1024 may be programmed to correspond to a specific neurotransmitter that the user is prompted to visualize. The circle 1024 may also be programmed to change color as the user is instructed to visualize a different neurotransmitter. As a non-limiting example, the circle 1024 may be red when the user is instructed to visualize the excitatory neurotransmitter dopamine and blue when the user is instructed to visualize the excitatory neurotransmitter oxytocin. The circle 1024 may also be programmed to brighten and oscillate to enable visualization of neural firing and connectivity. As previously discussed, the user may simultaneously be present with auditory and physical stimuli corresponding to scene 1020 illustrated in FIG. 10A.

FIG. 10B illustrates an exemplary scene depicted in the user's visual display for the oxygenation reset phase of treatment. In this non-limiting exemplary scene 1030, the user's display depicts a beach 1032 with a balloon 1034 floating in the distance. From the first-person perspective, the scene depicts the user on the beach 1032. The user is instructed to engage in controlled, forceful expiration to inflate the balloon 1034. As the user exhales, the balloon 1034 inflates. This exemplary exercise allows a user to visualize their controlled, deep breathing within the virtual reality environment, regulate blood pressure and heart rate, and increase circulation and oxygenation. To improve immersion within the virtual reality environment, the user may be presented with auditory and physical stimuli corresponding to scene 1030.

FIG. 10C illustrates an exemplary scene depicted in the user's visual display for the activity phase of treatment. In this non-limiting exemplary scene 1040, the user's display depicts a hanging punching bag 1044. From the first-person perspective, the scene depicts the user inside the boxing ring 1042 wearing boxing gloves 1048a, 1048b. The punching bag 1044 includes targets 1046a, 1046b, 1046c, 1046d, 1046e displaying various letters. A test object 1050 is displayed to the user. The user is instructed to use the input controllers 506a, 506b illustrated in FIG. 5 to sequentially punch the appropriate targets 1046a, 1046b, 1046c, 1046d, 1046e to spell the word depicted by the test object 1050. In the non-limiting embodiment illustrated in FIG. 10C, the test object 1050 is a cat. The punching bag 1044 features targets 1046a, 1046b, 1046c, 1046d, 1046e with the letters C, A, T, D, and K. To complete the exercise, a user would use the input controllers 506a, 506b to sequentially punch targets 1046d, 10446e, 1046b displaying the letters C, A, and T. Data corresponding to the user's inputs, such as the strength, speed, and accuracy of each action, is collected and stored. This data can be evaluated during the evaluation and modification step (discussed below) to measure the user's changing cognitive abilities. This data can also be used to present artificial intelligence-generated feedback to the user during and after treatment.

FIG. 10D illustrates another exemplary scene depicted in the user's visual display for the activity phase of treatment. In this non-limiting exemplary scene 1060, the user's display depicts a hanging punching bag 1064. In this non-limiting exemplary scene 1060, the punching bag 1064 can feature targets 1066a, 1066b, 1066c, 1066d with various numbers. From the first-person perspective, the scene depicts the user inside the boxing ring 1062 wearing boxing gloves 1068a, 1068b. A target math problem 1070 may be displayed to the user. The user may be instructed to use the input controllers 506a, 506b illustrated in FIG. 5 to punch the target 1066a, 1066b, 1066c, 1066d featuring the number that solves the presented math problem 1070. In the non-limiting embodiment illustrated in FIG. 10D, the math problem 1070 is 4+4. The punching bag 1064 features targets 1066a, 1066b, 1066c, 1066d with the numbers 2, 6, 7, and 8. To complete the exercise, a user would use the input controllers 506a, 506b to punch a target 1066c displaying the number 8. Data corresponding to the accuracy of the user's input may be collected and stored. This data can be evaluated during the evaluation and modification step (discussed below) to measure the user's changing cognitive abilities. This data can also be used to present artificial intelligence-generated feedback to the user during and after treatment.

FIG. 10E illustrates an exemplary scene depicted in the user's visual display for measuring the user's well-being at the beginning and end of each treatment session. In this non-limiting exemplary scene 1080, the user's display depicts a boxing ring 1082 with a hanging punching bag 1084 and a trainer avatar 1086 in a gym. The user's display may also present a series of interactive prompts 1088a, 1088b, 1088c for measuring the user's mental, emotional, and pain status. For example, the user's display may depict a mental state prompt 1088a, an emotional condition prompt 1088b, and a pain level prompt 1088c. In the non-limiting example illustrated in FIG. 10E, the prompts 1088 a, 1088 b, 1088 c include a movable scale ranging from 1-5. The user can be instructed to respond to the prompts 1088a, 1088b, 1088c using input controllers 506a, 506b illustrated in FIG. 5. For example, the user can be instructed to indicate their mental state by using the input controllers 506a, 506b to select a number along the continuum presented in the mental state prompt 1088a. User input data can be collected and stored for evaluation and modification.

FIG. 11 is a diagram of the treatment step of a process for improving brain health in a virtual reality environment. In the non-limiting diagram 1100, each point of the triangle represents a key component of the treatment step in accordance with The Harkins Method, Neural Network of Change. Each component sets the stage for the next, creating a continuous progression toward improved brain health. Diagram 1100 begins with mind preparation 1102, wherein circulation, oxygenation, and neurotransmitter levels are reset and balanced to increase neural activity 1104. Circulation and oxygenation increase neural activity 1104 by reducing oxidative stress, promoting cellular repair, and enhancing neuroplasticity. Restoration of the excitatory-inhibitory balance increases neural activity 1104 by maintaining neural firing and inducing neuroplasticity. Increased neural activity 1104 improves memory, decision-making, and mental resilience and sets the stage for improved neural firing and connection. The mind preparation 1102 component of treatment includes the neurotransmitter balance phase 1002 and oxygenation reset phase 1004 described in FIG. 10. To achieve the mind preparation 1102 component, visual, auditory, and physical stimuli may be presented to the user to restore the balance of excitatory and inhibitory neurotransmitters and increase oxygenation. Beginning with visual stimuli, the user may be guided through a virtual reality environment designed to enable visualization of neural firing and connectivity, neurotransmitter balance, and encourage deep, controlled breathing. The user may also be presented with auditory stimuli corresponding to the visual stimuli presented to the user. For example, the user may be presented with simultaneous auditory stimuli of slightly different frequencies to create binaural beats and stimulate brain activity. Other examples of auditory stimuli that can be presented to the user to achieve the mind preparation 1102 component of treatment include but are not limited to nature sounds, light classical music, and sounds or music embedded with delta or theta waves to support neuroplasticity. Physical stimuli administered through the input controllers 506a, 506b illustrated in FIG. 5 may also be used to achieve the mind preparation component 1102 of treatment. For example, haptic vibration corresponding to the visual and auditory stimuli may be sent to the input controllers 506a, 506b. The mind preparation 1102 component of treatment may also be achieved through guided meditation, mindfulness exercises, breathwork, and positive affirmations.

The next component is brain priming 1106, wherein the neurophysiological environment is conditioned to foster the formation of new neurons (neurogenesis) and neural connectivity 1108. The brain priming 1106 component focuses on executive functioning, speech, communication, time management and organization, reading and writing, math, spelling, vocabulary, word recall, and sentence structure. Brain priming 1106 increases neural firing in brain regions involved in learning, memory, and problem-solving, and prompts generation of new neurons to meet the increased processing demand. Increased neural firing and neurogenesis enhance neural connectivity 1108 by strengthening existing connections and integrating new neurons into existing neural networks, thereby improving communication between neurons. Brain priming 1106 sets the stage for the strengthening and formation of neural pathways.

The third component of a process for improving brain health in a virtual reality environment is body performance 1110, wherein repeated physical experiences form and strengthen neural pathways 1112. The body performance 1110 component focuses on rebuilding strength, mobility, and coordination. Body performance 1110 promotes communication between the brain and the muscles, as well as communication between areas of the brain responsible for physical activity like the motor cortex and the cerebellum. Increased neural communication generates and strengthens neural pathways 1112 by causing repeated connection between the same neurons, thereby improving both cognitive and physical aspects of brain health. The brain priming 1106 and the body performance 1110 components are realized during the activity 1006 phase of treatment described in FIG. 10.

FIG. 12 is a block diagram of the evaluation and modification step of a process for improving brain health in a virtual reality environment. During evaluation and modification, user input data 1202 collected and stored during treatment is delivered to an artificial intelligence processing unit 1204. Using a machine learning model, the artificial intelligence processing unit 1204 performs various functions to evaluate user input data from the previous treatment session. Examples of artificial intelligence functions include but are not limited to comparison to user input from other treatment sessions, comparison to average user input adjusted for age, injury severity, and treatment duration, and pattern recognition. Using this information, the artificial intelligence processing unit 1204 generates a modified treatment plan 1206 to optimize the improvement of brain health in a virtual reality environment. As depicted in FIG. 7, the modified treatment plan is implemented in the subsequent treatment session, wherein additional user input data 1202 is collected and stored for subsequent delivery to the artificial intelligence processing unit 1204. Continued treatment according to this personalized, adaptive treatment plan leads to strengthened neural pathways and improved brain health. The artificial intelligence processing unit 1204 can also generate treatment session feedback 1208 which can be presented to the user in the virtual reality environment during and after treatment.

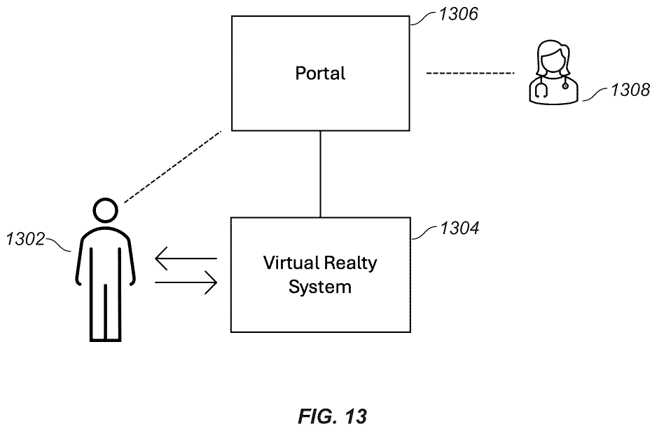

FIG. 13 is a block diagram of a virtual reality system in accordance with an illustrative embodiment. As previously discussed, the virtual reality system 1302 presents stimuli to the user 1302, collects, stores, and evaluates user input data, and generates personalized treatment plans. The virtual reality system 1302 may also include a portal 1306 for user 1304 and clinician 1308 access to information including but not limited to user input data and treatment session feedback. Using the portal 1306, the clinician 1308 may also manage the treatment plan.

While this disclosure has been particularly shown and described with reference to preferred embodiments, it will be understood by those skilled in the pertinent field of art that various changes in form and detail may be made therein without departing from the spirit and scope of the invention. The inventors expect skilled artisans to employ such variations as appropriate, and the inventors intend the invention to be practiced otherwise than as specifically described herein. Accordingly, this disclosure includes all modifications and equivalents of the subject matter recited in the claims appended hereto, as permitted by applicable law. Moreover, any combination of the above-described elements in all possible variations thereof is encompassed by the disclosure unless otherwise indicated herein or otherwise clearly contradicted by context.

Also, while various embodiments in accordance with the principles disclosed herein have been described above, it should be understood that they have been presented by way of example only, and not limitation. Thus, the breadth and scope of this disclosure should not be limited by any of the above-described exemplary embodiments, but should be defined only in accordance with any claims and their equivalents issuing from this disclosure. Furthermore, the above advantages and features are provided in described embodiments, but shall not limit the application of such issued claims to processes and structures accomplishing any or all of the above advantages.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically, and by way of example, although the headings refer to a “Technical Field,” the claims should not be limited by the language chosen under this heading to describe the so-called field. Further, a description of a technology as background information is not to be construed as an admission that certain technology is prior art to any embodiment(s) in this disclosure. Neither is the “Summary” to be considered as a characterization of the embodiment(s) set forth in issued claims. Furthermore, any reference in this disclosure to “invention” in the singular should not be used to argue that there is only a single point of novelty in this disclosure. Multiple embodiments may be set forth according to the limitations of the multiple claims issuing from this disclosure, and such claims accordingly define the embodiment(s), and their equivalents, that are protected thereby. In all instances, the scope of such claims shall be considered on their own merits in light of this disclosure, but should not be constrained by the headings set forth herein.

Moreover, the Abstract is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the claims reflect, inventive subject matter lies in less than all features of a single disclosed embodiment. Thus, the following claims are hereby incorporated into the Detailed Description, with each claim standing on its own as a separate embodiment.

Any and all publications, patents, and patent applications cited in this disclosure are herein incorporated by reference as if each were specifically and individually indicated to be incorporated by reference and set forth in its entirety herein.

What is claimed is:

1. A system for improving brain health using a virtual reality environment, comprising:
a virtual reality appliance having at least one visualization screen and at least one input controllable by a user
an artificial intelligence enabled program for providing and customizing the virtual reality environment with a treatment plan, including:
an initial testing phase, wherein the user's baseline metrics are measured,
a treatment plan development phase, wherein an initial treatment plan is created based on the user's baseline metrics,
a treatment phase, wherein a user is presented with stimuli within a virtual reality environment according to a treatment plan and user input data is collected and stored, and
an evaluation and modification phase, wherein artificial intelligence evaluates user input data and generates a modified treatment plan to optimize improvement of brain health.

2. The system of claim 1, wherein the virtual reality appliance is configured to deliver visual, auditory, and physical stimuli.

3. The system of claim 1, wherein the initial treatment phase is completed in a virtual reality environment.

4. The system of claim 1, wherein the treatment development phase includes comparison of the user's baseline metrics to average metrics.

5. The system of claim 4, wherein the treatment development phase is completed by artificial intelligence.

6. The system of claim 1, wherein the treatment phase includes:
a neurochemical balance phase, wherein the user is presented with stimuli configured to restore the balance of neurotransmitters to the normal range,
an oxygenation reset phase, wherein the user is presented with stimuli configured to improve circulation and oxygen intake, and
an activity phase, wherein the user is presented with physical and cognitive exercises within the virtual reality environment.

7. The system of claim 1, wherein the treatment phase includes:
a mind preparation component, wherein mindfulness exercises presented in the virtual reality environment condition the neurophysiological environment to increase neural activity,
a brain priming component, wherein cognitive exercises presented in the virtual reality environment condition the neurophysiological environment to foster neural connection, and
a body performance component, wherein physical exercises presented in the virtual reality environment strengthen neural pathways.

8. The system of claim 1, wherein an artificial intelligence processing unit evaluates user input data and generates a modified treatment plan during the evaluation and modification phase.

9. The system of claim 1, wherein the treatment phase includes presentation of stimuli in a virtual reality environment according to a modified treatment plan.

10. The system of claim 1, wherein the treatment phase and the evaluation and modification phase are alternately repeated according to a modified treatment plan.

11. A method of improving brain health in a virtual reality environment comprising the steps of:
an initial testing phase, wherein the user's baseline metrics are measured,
a treatment plan development phase, wherein a treatment plan is created based on the user's baseline metrics,
a treatment phase, wherein a user is presented with stimuli within a virtual reality environment according to a treatment plan and user input data is collected and stored, and
an evaluation and modification phase, wherein artificial intelligence evaluates user input data and modifies the treatment plan to optimize improvement of brain health.

12. The method of claim 11, wherein the initial treatment phase comprises presenting the user with exercises in a virtual reality environment.

13. The method of claim 12, wherein the treatment plan development phase comprises comparing the user's baseline metrics to average metrics.

14. The method of claim 13, wherein the treatment phase is completed by artificial intelligence.

15. The method of claim 13, wherein the treatment phase comprises:
a neurochemical balance phase, comprising presenting the user with stimuli configured to restore the balance of neurotransmitters to the normal range,
an oxygenation reset phase, comprising presenting the user with stimuli configured to improve circulation and oxygen intake, and
an activity phase, comprising engaging the user with physical and cognitive exercises within the virtual reality environment.

16. The method of claim 15, wherein the treatment phase comprises:
a mind preparation component, comprising presenting the user with mindfulness exercises in the virtual reality environment to condition the neurophysiological environment for increased neural activity,
a brain priming component, comprising presenting the user with cognitive exercises in the virtual reality environment to condition the neurophysiological environment for neuron connection, and
a body performance component, comprising presenting the user with physical exercises in the virtual reality environment to condition the neurophysiological environment for strengthening neural pathways.

17. The method of claim 15, wherein the evaluation and modification phase comprises an artificial intelligence processing unit evaluating user input data and generating a modified treatment plan.

18. The method of claim 17, wherein the treatment phase comprises presenting stimuli in a virtual reality environment according to a modified treatment plan.

19. The method of claim 18, wherein the treatment phase and evaluation and modification phases are alternately repeated according to a modified treatment plan.
